(19) United States
(12) Patent Application Publication
(10) Pub. No.: US 2024/0312365 A1
(43) Pub. Date: Sep. 19, 2024

(54) GUIDE ROPE

(71) Applicant: NIPPON TELEGRAPH AND TELEPHONE CORPORATION, Tokyo (JP)

(72) Inventors: Kakagu KOMAZAKI, Tokyo (JP); Junji WATANABE, Tokyo (JP)

(73) Assignee: NIPPON TELEGRAPH AND TELEPHONE CORPORATION, Tokyo (JP)

(21) Appl. No.: 18/576,091

(22) PCT Filed: Jul. 7, 2021
(86) PCT No.: PCT/JP2021/025588

§ 371 Date: Jan. 2, 2024

(51) Int. Cl. G09B 21/00; A61F 9/08; A63K 3/00

(52) U.S. Cl. CPC G09B 21/003; A61F 9/08; A63K 3/00

(57) ABSTRACT

To allow users to communicate with each other using the tactile sense, which is not limited to the direction and magnitude of force. A guide rope is constituted by a first gripping component, a first connection component, a second gripping component, and a second connection component that are annularly connected in sequence. An inner portion of the first gripping component, an inner portion of the first connection component, an inner portion of the second gripping component, and an inner portion of the second connection component are all hollow, and the inner portions of the components are connected to form one sealed space. The first gripping component and the second gripping component are formed in such a way as to be easily deformed by a change in pressure of air in the inner portion or a change in force from outside. The first connection component and the second connection component are formed in such a way as not to be easily deformed by a change in pressure of air in the inner portion, a change in force from outside, or a change in relative positional relationship between the users.

### TECHNICAL FIELD

The present invention relates to a guide rope used by a guide to guide a visually impaired person, for example, when an escort runner guides a visually impaired person in a marathon.

### BACKGROUND ART

Technologies that use a tactile sense to guide a visually impaired person include a technology of Non Patent Literature 1. Non Patent Literature 1 discloses an escort guide rope in which the rope has an annular shape. An escort runner and a visually impaired person hold the escort guide rope so that the users can communicate with each other using the direction and magnitude of force, for example, the escort runner can instruct the visually impaired person which direction to go.

Technologies for mutual communication using the tactile sense include a technology of Non Patent Literature 2. Non Patent Literature 2 discloses a communication tool in which one air chamber is formed by connecting a plurality of rubber balls and rubber air tubes so as to form an annular shape. When one person increases strength of holding one of the balls of the communication tool of Non Patent Literature 2, air is sent via an air tube to another ball being held by another person, and the ball being held by the other person bulges or starts to bulge. Thus, using the communication tool of Non Patent Literature 2 allows users to communicate with each other using the tactile sense.

### CITATION LIST

### Non Patent Literature

Non Patent Literature 1: “Guide to Guide Running (Basics)”, [online], Japan Blind Marathon Association, [searched on Apr. 15, 2021], the Internet <URL: https://jbma.or.jp/challenge/guiderunnner/preparation/>
Non Patent Literature 2: “Communication by Air”, [online], NTT INTERCOMMUNICATION CENTER, [retrieved on Apr. 15, 2021], the Internet <URL: https://www.ntticc.or.jp/ja/archive/works/communication-by-air/>

### SUMMARY OF INVENTION

### Technical Problem

The escort guide rope of Non Patent Literature 1 allows users to communicate with each other using the direction and magnitude of force. However, the escort guide rope allows for communication using only the direction and magnitude of force, and this poses a problem in that information that can be transmitted is limited.

The technology of Non Patent Literature 2 allows users to communicate with each other using the tactile sense. However, a flexible rubber air tube is used, and this poses a problem in that it is difficult to communicate using the direction and magnitude of force, and usage as a guide rope is not possible.

It is an object of the present invention to provide a guide rope that allows users to communicate with each other using the tactile sense, which is not limited to the direction and magnitude of force.

### Solution to Problem

In order to solve the above problems, one aspect of the present invention provides a guide rope used for communication between two users. In the guide rope, a first gripping component to be gripped by a first user among the two users, a second gripping component to be gripped by a second user among the two users, a first connection component, and a second connection component are annularly connected, each of the first gripping component and the second gripping component is a hollow three-dimensional object having an outer shell provided with two connection portions (hereinafter referred to as a “first connection portion” and a “second connection portion”), each of the first connection component and the second connection component is a tube provided with two ends (hereinafter referred to as a “first end” and a “second end”), the first connection portion of the first gripping component is connected to the first end of the first connection component, the second connection portion of the first gripping component is connected to the second end of the second connection component, the first connection portion of the second gripping component is connected to the first end of the second connection component, the second connection portion of the second gripping component is connected to the second end of the first connection component, one sealed space is formed by a hollow inner portion of the first gripping component, a hollow inner portion of the first connection component, a hollow inner portion of the second gripping component, and a hollow inner portion of the second connection component, the outer shell of each of the first gripping component and the second gripping component is formed in such a way as to be easily deformed by any of a change in pressure of air in the inner portion and a change in force applied from outside, and an outer shell of each of the first connection component and the second connection component is formed in such a way as not to be easily deformed by any of a change in pressure of air in the inner portion and a change in force applied from outside, and not to be easily deformed by a change in relative positional relationship between the first user gripping the first gripping component and the second user gripping the second gripping component.

### Advantageous Effects of Invention

The guide rope of the present invention allows users to communicate with each other using the tactile sense, which is not limited to the direction and magnitude of force.

### BRIEF DESCRIPTION OF DRAWINGS

FIG. 1 illustrates an external appearance of a guide rope 100.

FIG. 2A is a plan view illustrating the guide rope 100. FIG. 2B is a side view illustrating the guide rope 100.

FIG. 3A is a schematic cross-sectional view for illustrating a configuration of a gripping component 110-i.

FIG. 3B is a schematic side view for illustrating the configuration of the gripping component 110-i.

FIG. 4A is a schematic cross-sectional view for illustrating a configuration of a connection component 120-j. FIG. 4B is a schematic side view for illustrating the configuration of the connection component 120-j.

FIG. 5 is a schematic cross-sectional view for illustrating how the components are connected in the guide rope 100.

### DESCRIPTION OF EMBODIMENTS

### First Embodiment

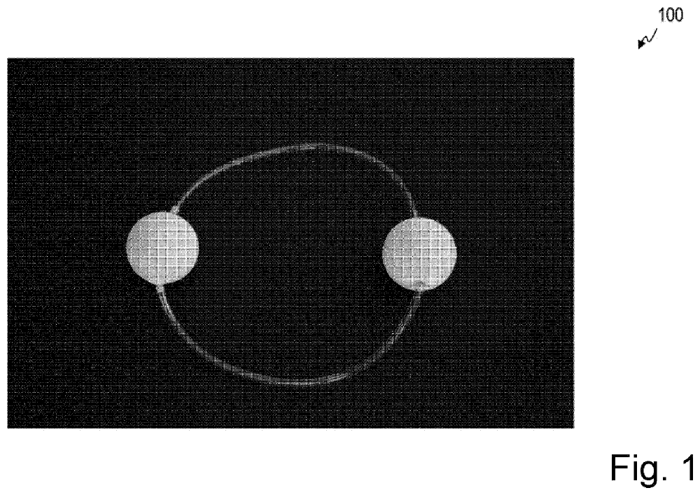

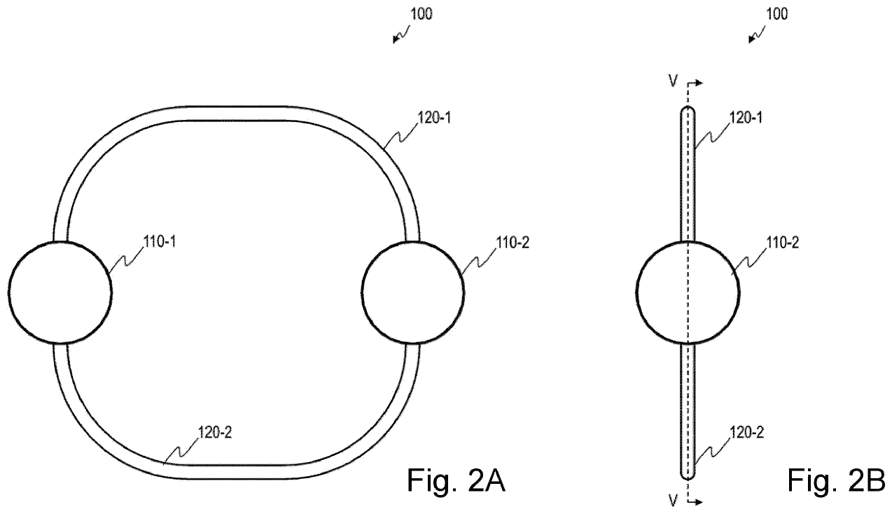

A guide rope 100 of a first embodiment is a three-dimensional object illustrated in FIGS. 1 and 2, and includes a first gripping component 110-1, a second gripping component 110-2, a first connection component 120-1, and a second connection component 120-2 as illustrated in FIG. 2A. More specifically, the guide rope 100 is constituted by the first gripping component 110-1, the first connection component 120-1, the second gripping component 110-2, and the second connection component 120-2 that are annularly connected in sequence. The first gripping component 110-1 is gripped by one user, who is one of two users: a visually impaired person and a guide (the one user is hereinafter referred to as a “first user”). The second gripping component 110-2 is gripped by the other user, who is not the first user but is the other of the two users: the visually impaired person and the guide (the other user is hereinafter referred to as a “second user”). The guide may be a sighted person, or may be a visually impaired person.

### [First Gripping Component 110-1 and Second Gripping Component 110-2]

The first gripping component 110-1 and the second gripping component 110-2 have the same structure, and thus will be described below as gripping components 110-i, in which i represents 1 in a case of the first gripping component 110-1, and i represents 2 in a case of the second gripping component 110-2.

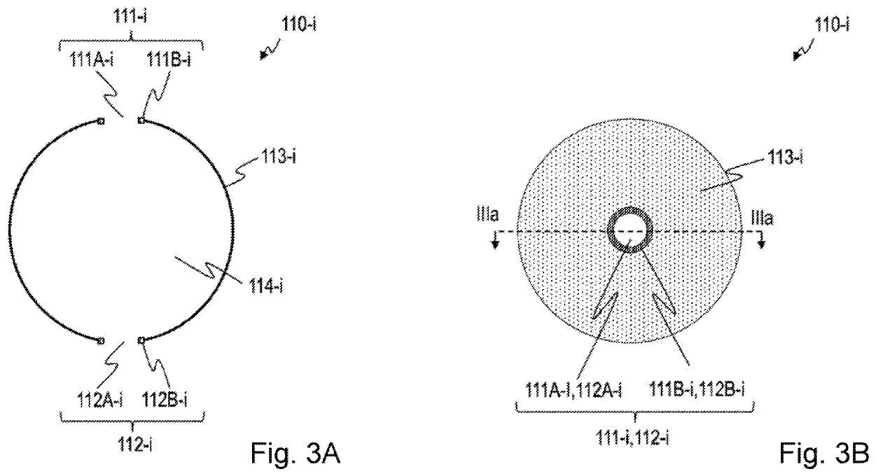

FIG. 3A is a schematic cross-sectional view taken along broken line IIIa-IIIa in FIG. 3B. As illustrated in the cross-sectional view of FIG. 3A, the gripping component 110-i is a hollow three-dimensional object having an outer shell 113-i provided with two connection portions: a first connection portion 111-i and a second connection portion 112-i. The hollow portion of the gripping component 110-i is hereinafter referred to as an inner portion 114-i. The outer shell 113-i forms a three-dimensional object when the inner portion 114-i is filled with gas. The shape of the three-dimensional object is optional, and may be a sphere, a spheroid, or any other shape. The size of the three-dimensional object is optional as long as the size allows for gripping by a user, and, for example, the volume including the gas with which the inner portion 114-i is filled may be approximately 150 cm³, and in a case where the three-dimensional object is a sphere, the diameter may be approximately 6.6 cm. The outer shell 113-i is formed to be easily deformed by any of a change in pressure of the gas in the inner portion 114-i and a change in force applied from the outside. That is, the outer shell 113-i may be formed by using an elastic body, and may be formed by using, for example, a thin rubber sheet having low hardness. An example of the thin rubber sheet having low hardness is a rubber (natural rubber or synthetic rubber) sheet of approximately 1 mm in thickness. More specifically, for example, a soft tennis ball provided with the first connection portion 111-i and the second connection portion 112-i may be used as the gripping component 110-i.

Each of the first connection portion 111-i and the second connection portion 112-i may be provided so as to include a point where a straight line passing through the center of gravity of the three-dimensional object is in contact with the outer shell 113-i. In a case where a soft tennis ball provided with the first connection portion 111-i and the second connection portion 112-i is used as the gripping component 110-i, the shape of the three-dimensional object when the inner portion 114-i is filled with gas is a sphere and the center of gravity of the three-dimensional object is the center of the sphere, and thus, each of the first connection portion 111-i and the second connection portion 112-i may be provided so as to include a point where a straight line passing through the center of the sphere is in contact with the outer shell 113-i.

FIG. 3B is a side view of the gripping component 110-i as viewed from the first connection portion 111-i side and the gripping component 110-i as viewed from the second connection portion 112-i side. The first connection portion 111-i is provided with an opening 111A-i and a joint portion 111B-i. The second connection portion 112-i is provided with an opening 112A-i and a joint portion 112B-i.

### [First Connection Component 120-1 and Second Connection Component 120-2]

The first connection component 120-1 and the second connection component 120-2 have the same structure, and thus will be described below as connection components 120-j, in which j represents 1 in a case of the first connection component 120-1, and j represents 2 in a case of the second connection component 120-2.

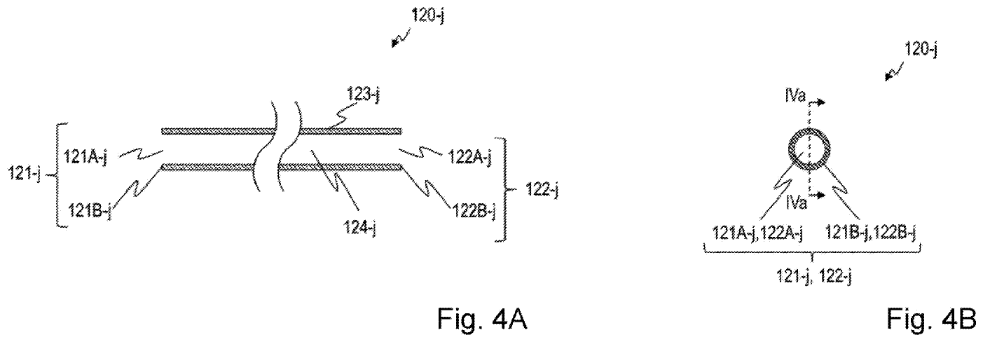

FIG. 4A is a schematic cross-sectional view taken along broken line IVa-IVa in FIG. 4B. As illustrated in the cross-sectional view of FIG. 4A, the connection component 120-j is a tube provided with two ends: a first end 121-j and a second end 122-j. Hereinafter, the tube portion of the connection component 120-j is referred to as an outer shell 123-j, and a hollow portion of the connection component 120-j is referred to as an inner portion 124-j. FIG. 4B is a side view of the connection component 120-j as viewed from the first end 121-j side and the connection component 120-j as viewed from the second end 122-j side. The first end 121-j of the connection component 120-j is constituted by an opening 121A-j, which is an end of the hollow inner portion 124-j, and a joint portion 121B-j, which is an end of the outer shell 123-j. The second end 122-j of the connection component 120-j is constituted by an opening 122A-j, which is an end of the hollow inner portion 124-j, and a joint portion 122B-j, which is an end of the outer shell 123-j.

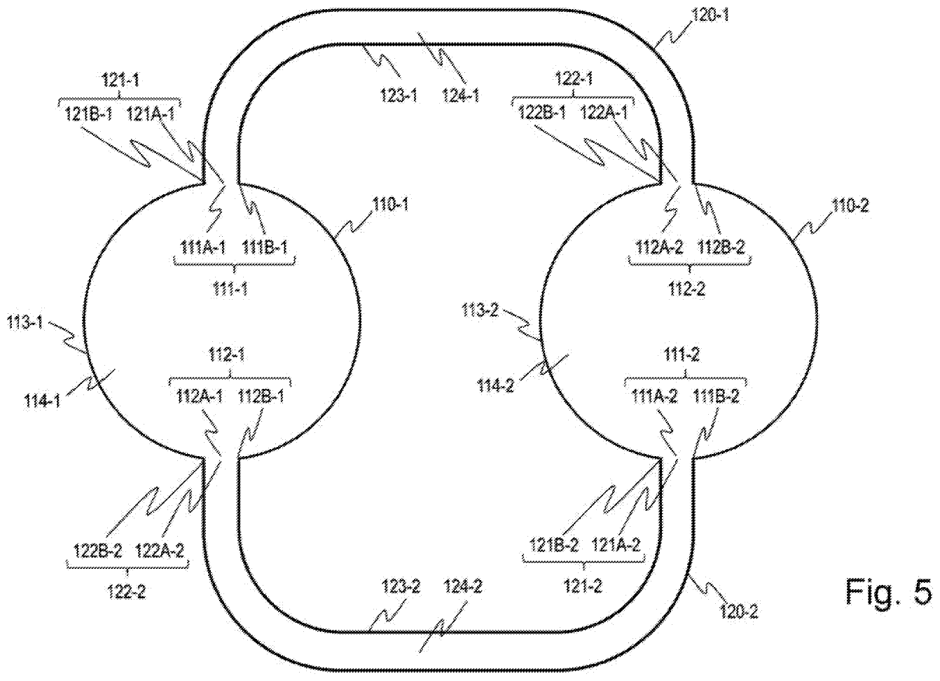

FIG. 5 is a cross-sectional view schematically illustrating how the first gripping component 110-1, the first connection component 120-1, the second gripping component 110-2, and the second connection component 120-2 are connected in the guide rope 100, and is a schematic cross-sectional view taken along broken line V-V in FIG. 2B. FIG. 2B is a side view of the guide rope 100 as viewed from the second gripping component 110-2 side. A first end 121-1 of the first connection component 120-1 is connected to a first connection portion 111-1 of the first gripping component 110-1, a second end 122-1 of the first connection component 120-1 is connected to a second connection portion 112-2 of the second gripping component 110-2, a first end 121-2 of the second connection component 120-2 is connected to a first connection portion 111-2 of the second gripping component 110-2, and a second end 122-2 of the second connection component 120-2 is connected to a second connection portion 112-1 of the first gripping component 110-1. With these four connections, the first gripping component 110-1, the first connection component 120-1, the second gripping component 110-2, and the second connection component 120-2 are annularly connected in sequence.

The length of the connection component 120-j is optional as long as two users can individually grip the gripping components 110-i without being in contact with each other, and may be, for example, approximately 30 cm. The inner diameter of the tube is optional as long as the inner diameter does not obstruct a flow of air generated by a change in pressure outside the outer shell 113-i of the gripping component 110-i or does not provide a high resistance to the flow of air, and may be, for example, approximately 4 mm. The outer shell 123-j is formed so as not to be easily deformed by any of a change in pressure of gas inside and a change in force applied from the outside, and so as not to be easily deformed by a change in relative positional relationship between the first user who is gripping the first gripping component and the second user who is gripping the second gripping component, and is formed by using, for example, soft polyvinyl chloride (soft PVC) of approximately 2 mm in thickness. That is, for example, a tube that is approximately 30 cm in length, approximately 4 mm in inner diameter, and approximately 6 mm in outer diameter, and is formed by using soft PVC can be used as the connection component 120-j.

### [Connection]

Details of how the first gripping component 110-1, the first connection component 120-1, the second gripping component 110-2, and the second connection component 120-2 are connected in the guide rope 100 will be described with reference to FIG. 5.

At the connection between the first connection portion 111-1 of the first gripping component 110-1 and the first end 121-1 of the first connection component 120-1, a joint portion 111B-1 of the first connection portion 111-1 of the first gripping component 110-1 and a joint portion 121B-1 of the first end 121-1 of the first connection component 120-1 are joined, and by this joining, an opening 111A-1 of the first connection portion 111-1 of the first gripping component 110-1 and an opening 121A-1 of the first end 121-1 of the first connection component 120-1 are connected.

At the connection between the second connection portion 112-1 of the first gripping component 110-1 and the second end 122-2 of the second connection component 120-2, a joint portion 112B-1 of the second connection portion 112-1 of the first gripping component 110-1 and a joint portion 122B-2 of the second end 122-2 of the second connection component 120-2 are joined, and by this joining, an opening 112A-1 of the second connection portion 112-1 of the first gripping component 110-1 and an opening 122A-2 of the second end 122-2 of the second connection component 120-2 are connected.

At the connection between the first connection portion 111-2 of the second gripping component 110-2 and the first end 121-2 of the second connection component 120-2, a joint portion 111B-2 of the first connection portion 111-2 of the second gripping component 110-2 and a joint portion 121B-2 of the first end 121-2 of the second connection component 120-2 are joined, and by this joining, an opening 111A-2 of the first connection portion 111-2 of the second gripping component 110-2 and an opening 121A-2 of the first end 121-2 of the second connection component 120-2 are connected.

At the connection between the second connection portion 112-2 of the second gripping component 110-2 and the second end 122-1 of the first connection component 120-1, a joint portion 112B-2 of the second connection portion 112-2 of the second gripping component 110-2 and a joint portion 122B-1 of the second end 122-1 of the first connection component 120-1 are joined, and by this joining, an opening 112A-2 of the second connection portion 112-2 of the second gripping component 110-2 and an opening 122A-1 of the second end 122-1 of the first connection component 120-1 are connected.

The openings are connected as described above, and thus one sealed space is formed by a hollow inner portion 114-1 of the first gripping component 110-1, a hollow inner portion 124-1 of the first connection component 120-1, a hollow inner portion 114-2 of the second gripping component 110-2, and a hollow inner portion 124-2 of the second connection component 120-2. The inside of the formed sealed space is filled with gas. The gas with which the sealed space is filled may be any gas as long as the gas does not erode or degrade the gripping components 110-i and the connection components 120-j, and, for example, air may be used. The air pressure inside the sealed space may be set to such an extent that the above-described shape can be maintained when an outer shell 113-1 of the first gripping component 110-1 and an outer shell 113-2 of the second gripping component 110-2 are individually gripped by users.

### [Usage]

The guide rope 100 is used in a state where the first user is gripping the first gripping component 110-1 and the second user is gripping the second gripping component 110-2. When the first user increases the strength of holding the first gripping component 110-1 the first user is gripping, a part of gas in the inner portion 114-1 of the first gripping component 110-1 is pushed out into the inner portion 124-1 of the first connection component 120-1 and the inner portion 124-2 of the second connection component 120-2, gas is sent from the inner portion 124-1 of the first connection component 120-1 and the inner portion 124-2 of the second connection component 120-2 into the inner portion 114-2 of the second gripping component 110-2, and thus the outer shell 113-2 of the second gripping component 110-2 bulges or starts to bulge, thereby allowing the second user to perceive via the tactile sense that the first user has increased the strength of holding the first gripping component 110-1. Similarly, when the second user increases the strength of holding the second gripping component 110-2, the outer shell 113-1 of the first gripping component 110-1 bulges or starts to bulge, and the first user can perceive via the tactile sense that the second user has increased the strength of holding the second gripping component 110-2. When the first user applies a pulling force in a certain direction to the first gripping component 110-1 the first user is gripping, the pulling force is also applied in the certain direction to the second gripping component 110-2 connected by the first connection component 120-1 and the second connection component 120-2 so that the relative positional relationship with the first gripping component 110-1 does not easily change, and the second user can perceive via the tactile sense that the first user has applied the pulling force in the certain direction to the first gripping component 110-1. Similarly, when the second user applies a pulling force to the second gripping component 110-2 in a certain direction, the pulling force is applied to the first gripping component 110-1 in the certain direction, and the first user can perceive via the tactile sense that the pulling force has been applied in the certain direction. As described above, using the guide rope 100 allows the first user and the second user to perform mutual communication including information regarding a pulling direction, information regarding a pulling force, and information regarding holding, which is information regarding other than the force and the direction, without need for an electric control mechanism or power supply.

### [Experiment Results]

The inventors conducted an experiment with participants by using eight types of guide ropes 100, in which a soft tennis ball was used as the gripping component 110-i, and two types of angle formed by the first connection portion 111-i and the second connection portion 112-i of the gripping component 110-i, two types of hardness of the connection component 120-j, and two types of length of the connection component 120-j were set.

The two types of angle formed by the first connection portion 111-i and the second connection portion 112-i of the gripping component 110-i used in the experiment were 90 degrees and 180 degrees in central angle in a cross section of the sphere of the soft tennis ball. In the case of the gripping component 110-i with 90 degrees in central angle, a finger sometimes got caught on the connection component 120-j. On the other hand, in the case of the gripping component 110-i with 180 degrees in central angle, the gripping component 110-i has been found to be easier to hold, without a finger getting caught on the connection component 120-j. That is, it has been found that the first connection portion 111-i and the second connection portion 112-i of the gripping component 110-i are preferably provided individually at contact points between a straight line passing through the center of gravity of the three-dimensional object, that is, the gripping component 110-i, and the outer shell of the three-dimensional object.

The two types of hardness of the connection component 120-j used in the experiment were a soft tube and a hard tube as the connection component 120-j. The soft tube is a silicone tube of approximately 6 mm in outer diameter and approximately 4 mm in inner diameter, and the hard tube is a soft PVC tube of approximately 6 mm in outer diameter and approximately 4 mm in inner diameter. In the case of the guide rope 100 using the soft tube as the connection component 120-j, it has been found that the guide rope has problems such as being too soft and having too much play, not providing a secure feeling, being difficult to control, causing wide turn when a turn is made, and not allowing a feeling of making a turn to be easily perceived. On the other hand, in the case of the guide rope 100 using the hard tube as the connection component 120-j, it has been found that the guide rope has advantages such as providing a secure feeling, providing reliability, being stable, and being excellent in tension. That is, it has been found that the connection component 120-j is preferably formed so as not to be easily deformed by a change in relative positional relationship between the first user who is gripping the first gripping component 110-1 and the second user who is gripping the second gripping component 110-2, and, for example, it is preferable to use, as the connection component 120-j, a tube that is approximately 6 mm in outer diameter and approximately 4 mm in inner diameter, and is formed by using soft PVC.

The two types of length of the connection component 120-j used in the experiment were 15 cm and 30 cm. In the case of the connection component 120-j of 15 cm in length, it has been found that there are problems such as collision due to too little play, hands colliding with each other due to the length being too short, and difficulty in holding. On the other hand, in the case of the connection component 120-j of 30 cm in length, it has been found that there are advantages such as the distance being appropriate and the length being better than 15 cm when used for a novice or guidance. That is, it has been found that the length of the connection component 120-j is preferably approximately 30 cm.

### <Supplement>

The description of the embodiment of the present invention described above has been presented for purposes of illustration and description. There is no intention to be comprehensive or to limit the invention to the disclosed precise form. Modifications and variations can be made from the foregoing instructions. The embodiment has been selected and represented in order to provide the best illustration of the principles of the present invention and to enable those skilled in the art to utilize the present invention in various embodiments with various modifications added such that the present invention is appropriate for considered practical use. All such modifications and variations are within the scope of the present invention as defined by the appended claims, interpreted in accordance with a fairly and legally equitable breadth.

1. A guide rope used for communication between two users,
wherein a first gripping component to be gripped by a first user among the two users, a second gripping component to be gripped by a second user among the two users, a first connection component, and a second connection component are annularly connected,
each of the first gripping component and the second gripping component is a hollow three-dimensional object having an outer shell provided with two connection portions (hereinafter referred to as a “first connection portion” and a “second connection portion”),
each of the first connection component and the second connection component is a tube provided with two ends (hereinafter referred to as a “first end” and a “second end”),
the first connection portion of the first gripping component is connected to the first end of the first connection component,
the second connection portion of the first gripping component is connected to the second end of the second connection component,
the first connection portion of the second gripping component is connected to the first end of the second connection component,
the second connection portion of the second gripping component is connected to the second end of the first connection component,
one sealed space is formed by a hollow inner portion of the first gripping component, a hollow inner portion of the first connection component, a hollow inner portion of the second gripping component, and a hollow inner portion of the second connection component,
the outer shell of each of the first gripping component and the second gripping component is formed in such a way as to be easily deformed by any of a change in pressure of air in the inner portion and a change in force applied from outside, and
an outer shell of each of the first connection component and the second connection component is formed in such a way as not to be easily deformed by any of a change in pressure of air in the inner portion and a change in force applied from outside, and not to be easily deformed by a change in relative positional relationship between the first user gripping the first gripping component and the second user gripping the second gripping component.

2. The guide rope according to claim 1, wherein
the three-dimensional object constituting each of the first gripping component and the second gripping component is a substantially spherical object of approximately 6.6 cm in diameter.

3. The guide rope according to claim 2, wherein
in each of the first gripping component and the second gripping component, the first connection portion and the second connection portion are provided in such a way as to individually include contact points between a straight line passing through a center of gravity of the three-dimensional object and the outer shell of the three-dimensional object.

4. The guide rope according to claim 3, wherein
the outer shell of the three-dimensional object is formed by using a rubber sheet of approximately 1 mm in thickness.

5. The guide rope according to claim 3, wherein
each of the first connection component and the second connection component is approximately 30 cm in length.

6. The guide rope according to claim 5, wherein
each of the first connection component and the second connection component is approximately 6 mm in outer diameter and approximately 4 mm in inner diameter, and is formed by using soft PVC.

7. The guide rope according to claim 4, wherein
each of the first connection component and the second connection component is approximately 30 cm in length.

8. The guide rope according to claim 7, wherein
each of the first connection component and the second connection component is approximately 6 mm in outer diameter and approximately 4 mm in inner diameter, and is formed by using soft PVC.
